(19) United States
(12) Patent Application Publication
(10) Pub. No.: US 2022/0339100 A1
(43) Pub. Date: Oct. 27, 2022

(54) THERMOSENSITIVE MODIFIED CHITIN HYDROGEL LOCAL ANESTHETIC-LOADED SUSTAINED-RELEASE ANALGESIA SYSTEM, PREPARATION METHOD AND USE

(71) Applicant: Wuhan University, Wuhan (CN)

(72) Inventors: Xulin Jiang, Wuhan (CN); Qianqian Qiao, Wuhan (CN); Jieyu Zheng, Wuhan (CN); Yalan Zhong, Wuhan (CN)

(21) Appl. No.: 17/660,334

(22) Filed: Apr. 22, 2022

(30) Foreign Application Priority Data
Apr. 22, 2021 (CN) 202110437908.9

(51) Int. Cl. A61K 9/06; A61K 9/00; A61K 47/36; A61K 31/445

(52) U.S. Cl. CPC A61K 9/06; A61K 9/0019; A61K 47/36; A61K 31/445

(57) ABSTRACT

The invention discloses a thermosensitive modified chitin hydrogel local anesthetic-loaded sustained-release analgesia system, a preparation method and use. A thermosensitive chitin derivative is dissolved into water at a low temperature, a local anesthetic and analgesic drug aqueous solution or degradable polymer microspheres loaded with local anesthetic and analgesic drugs are added under a liquid state, the above materials are evenly mixed at a low temperature, the obtained mixture is injected into a site needing local anesthesia and analgesia so as to quickly form a gel under a body temperature and release the drug. The local anesthetic drug sustained-release analgesia composite hydrogel system is mainly characterized in that preparation is simple, organic solvents are not used, the local anesthetic and analgesic drug can be slowly released, and the problems that the existing local anesthetic drugs have a short action time are solved partly.

### CROSS REFERENCES TO RELATED APPLICATIONS

The present application claims foreign priority of Chinese Patent Application No. 202110437908.9, filed on Apr. 22, 2021 in the China National Intellectual Property Administration, the disclosures of all of which are hereby incorporated by reference.

### TECHNICAL FIELD

The disclosure belongs to the field of pharmaceutical preparations, relates to a drug sustained-release system as well as a preparation method and use thereof, more particularly to an injectable hydrogel local anesthetic-loaded sustained-release analgesia system, a preparation method and use.

### BACKGROUND OF THE DISCLOSURE

A pain is an undesirable sensory and emotional feeling accompanying with substantial or potential tissue damages. It is a subjective feeling, which is considered as the fifth largest vital sign after respiration, pulse, body temperature and blood pressure, and is one of the most common symptoms in clinic. The pain can be divided into an acute pain and a chronic pain. The acute pain occurs after trauma or surgery and has self limitation. The acute pain can be relieved after the tissue damage is recovered, and is developed into chronic pain if no relieving. The chronic pain refers to a pain whose lasting time exceeds normal healing time of an acute damage or disease and which is recurred for a long term. Some long-term violent pains, such as nerve pains, cancer pains, arthritis pains and lumbagos and backaches, are not only torments that are unbearable for patients but also can cause physiological dysfunction. Thus, analgesia is one of important tasks and difficulties in clinical treatment, and is also an important subject for medical researches.

At present, postoperative analgesia mainly includes systemic analgesia and local analgesia. The former mainly means oral or intravenous administration of analgesic drugs such as opioid drugs and nonsteroidal anti-inflammatory drugs (NSAIDs). Because these drugs have effects on multiple systems, especially many side effects such as constipation, nausea and vomiting, more seriously, respiratory depression and even death can be caused. In addition, long-term administration of opioid drugs can cause tolerance, drug abuse and addiction. The local analgesia mainly means local application of anaesthetic drugs (local anaesthetic drugs). The local anaesthetic drug can block the delivery of algesia signals to a central nervous system by blocking sodium and other ion channels on nerve cytomembranes so as to cause the block of electric signals of all structures at a nerve downstream, thereby achieving the effect of postoperative analgesia. The local anaesthetic drug has strong action pertinence, reduces systemic side effects and has better safety. Meanwhile, the local analgesia is simple in operation and can be completed in outpatient service, so it is cheap in cost and decreases the burden of patients. At present, the local anaesthetic drug has been applied to postoperative analgesia of surgical operations such as thoracotomy, laparotomy, cesarean section, breast cancer operation, cosmetic breast surgery and limb amputation, and plays an important role in controlling and relieving acute and chronic pains after various large-scale surgeries.

However, the existing local anaesthetic and analgesic drugs have a short action time, generally no more than a few hours, and the analgesia time required clinically is generally 24 hours, several days or more than ten days. Therefore, it is needed to increase drug dosage, repeated administration, in vivo implantation of a catheter, self control of an analgesia pump and other technologies to prolong its analgesia effect. However, cardio cerebral side effects of these anesthetic drugs have low incidence, but they can greatly threaten the life safety of patients once occurring, the implantation of the catheter and the self control of the analgesia pump not only needs expensive equipment and continuous guardianship which should be removed after use, but also easily causes obstruction and damage of the catheter, infection complications and other problems. Sustained-release local anesthetic drugs can not only improve the analgesic effect of the local anesthetic drugs, but also reduce the frequency of medication and reduce adverse reactions caused by high-dose use, so they have attracted more and more attentions. At present, a local anesthetic long-acting sustained-release administration system mainly includes microspheres, liposomes, implants, injectable in-situ gel, etc. Microspheres are spherical or quasi spherical tiny spherical entities. The particle size range of the microspheres is generally 1-500 microns, the small particle size can be several nanometers, and the large particle size can be up to 800 microns. The microspheres have better fluidity than irregular powder particles. The traditional preparation methods of drug-loaded microspheres mainly include an emulsification dispersion-solvent evaporation method, a spray drying method and a coagulation method. The emulsification dispersion method often needs to use organic solvents and surfactants, is simple and easy to operate, and is a method which is most frequently used in laboratory researches, but it has residual problems of organic solvents and dispersants and has many affection factors for scale-up production, so its process control requirements are relatively high. The spray drying method is simple in process, low in cost and easy in large-scale and continuous production, but its use is limited in laboratory researches, the size of microspheres is difficult to control, and the microspheres are easy to aggregate. The coagulation method means that the solubility changes of a material in a mixed solution due to influences from external physical and chemical factors such as opposite charges, dehydration and solvent replacement so that the material is precipitated from the solution. The microspheres prepared by these commonly used pelletizing drug-loaded methods has board size distribution, many drugs are deposited and crystallized on the surfaces of microspheres, and there is a phenomenon of high burst release.

An injectable in-situ gel can be administrated in a form of a flowable drug-containing solution. Through phase transformation or in-situ crosslinking reaction due to in-vivo physiological environment at the injection site, it is transferred from a liquid state to a non-crosslinked semisolid gel or a crosslinked gel, where the crosslinking herein can be physical crosslinking or chemical crosslinking. Because of the absence of chemical reactions during the gel formation, the gelation is fast, simpler and safer in application. Especially, thermosensitive injectable hydrogels can achieve solution-gel transition mainly based on change in external temperatures without chemical reagents, have good biocompatibility and are widely used for medical purposes. This kind of thermosensitive gels are maintained as a flowable liquid at a low temperature (4° C.), can homogeneously load cells/drugs and can be implanted without surgical operation so as to improve patient's compliance. After being injected into a body, the polymer solution can rapidly form a gel at a body temperature (37° C.) to avoid the loss of cells, bioactive drug molecules and others, and can be used as an in-situ drug delivery system to achieve local slow release of drugs. Because a temperature response is a response that is relatively easily achieved and is most effective, this kind of intelligent hydrogel drug sustained-release carriers have an attractive development prospect. It is very important to develop new sustained-release dosage forms of long-acting local anesthetic drugs which can prolong the action time, is convenient to use and good in degradability and compatibility.

### SUMMARY OF DISCLOSURE

In view of the deficiencies in the prior art in combination with the basis of previous works, the objective of the disclosure is to provide a thermosensitive modified chitin hydrogel local anesthetic-loaded sustained-release analgesia system, a preparation method and use.

According to the disclosure, thermosensitive modified chitin which is a flowing liquid at a low temperature, and has good mobility and injectablity can be quickly form a gel under the body temperature through physical crosslinking and release the drug slowly at the site, thereby prolonging the analgesia time of the local anesthetic drug after a local anesthetic and analgesic drug aqueous solution or degradable polymer microspheres loaded with local anesthetic and analgesic drugs are added and evenly mixed. The technical solution adopted by the disclosure is specifically as follows:

The disclosure provides a thermosensitive modified chitin hydrogel local anesthetic-loaded sustained-release analgesia system, wherein the local anesthetic-loaded sustained-release analgesia system is an injectable mixed hydrogel prepared by mixing thermosensitive modified chitin with a local anesthetic and analgesic drug, the gel transition temperature of the thermosensitive modified chitin is lower than a body temperature, the concentration of the thermosensitive modified chitin in the injectable mixed hydrogel is 0.5-5% by mass, and the concentration of the local anesthetic and analgesic drug in the injectable mixed hydrogel is 0.1-5% by mass.

Preferably, the thermosensitive modified chitin is a combination of any one or more of thermosensitive hydroxypropyl chitin, thermosensitive hydroxyethyl chitin or thermosensitive hydroxybutyl chitin.

Preferably, the local anesthetic-loaded sustained-release analgesia system also contains hyaluronic acid dissolved into the injectable mixed hydrogel to serve as an auxiliary ingredient.

Preferably, the mass fraction of hyaluronic acid in the local anesthetic-loaded sustained-release analgesia system is 0.1-1%.

Preferably, in the local anesthetic-loaded sustained-release analgesia system, the concentration of the thermosensitive hydroxypropyl chitin is 1-3% by mass, and the concentration of hyaluronic acid is 0.2-0.8% by mass, and the concentration of the local anesthetic and analgesic drug is 0.5-3% and the local anesthesia selected from bupivacaine hydrochloride or ropivacaine hydrochloride.

Preferably, the at least one drug substance is present in an amount sufficient to provide long lasting local analgesia, local anesthesia.

Preferably, the local anesthetic and analgesic drug is an amino amide anaesthetic, an amino ester anesthetics and mixtures thereof, selected from any one or more of procaine, bupivacaine, levobupivacaine, tetracaine, ropivacaine, etidocaine, articaine, lidocaine, mepivacaine, prilocaine and oxethazaine, and their salts and prodrugs.

Preferably, the local anesthetic-loaded sustained-release analgesia system also contains degradable polymer microspheres loaded with local anesthetic and analgesic drugs, which are evenly dispersed in the injectable mixed hydrogel.

Preferably, the degradable polymer microspheres are thermosensitive carboxymethyl chitin porous microspheres, and the loaded local anesthetic and analgesic drug is ropivacaine hydrochloride.

The disclosure also provides a preparation method of a thermosensitive modified chitin hydrogel local anesthetic-loaded sustained-release analgesia system, comprising the following steps:

(1) dissolving thermosensitive hydroxypropyl chitin and hyaluronic acid into normal saline or alkaline water below the gel transition temperature to obtain solution A;

(2) dissolving local anesthetic and analgesic drug hydrochloride into normal saline or acidic water to obtain solution B; and

(3) mixing the solution A with the solution B at a gel transition temperature to prepare a mixed hydrogel precursor solution, and adjusting pH to 5-6 to obtain a homogenous injectable mixed hydrogel, namely, the local anesthetic-loaded sustained-release analgesia system.

Preferably, the low temperature is 2-30° C., further preferably 4-15° C., adjusting pH to 5.5-6 to obtain a homogenous injectable mixed hydrogel, the gel transition temperature of the thermosensitive hydroxypropyl chitin is lower than a body temperature, and the injectable mixed hydrogel can be formed into a hydrogel sustained-release drug under the body temperature.

The disclosure also provides a preparation method of a thermosensitive modified chitin hydrogel/microsphere composite local anesthetic-loaded sustained-release analgesia system, comprising the following steps:

(1) dissolving thermosensitive modified chitin into normal saline or alkaline water below the low temperature to obtain solution A;

(2) preparing degradable polymer microspheres containing local anesthetic and analgesic drugs; and

(3) evenly mixing the degradable polymer microspheres containing the local anesthetic and analgesic drugs in the solution A at a low temperature to prepare an injectable mixed hydrogel, namely, the local anesthetic-loaded sustained-release analgesia system, wherein the low temperature is lower than the gel transition temperature of the thermosensitive modified chitin.

Preferably, hyaluronic acid is added in the process of preparing the solution A in step (1).

Preferably, the low temperature is 2-30° C., further preferably 4-15° C., adjusting pH to 5.5-6.0 to obtain a homogenous injectable mixed hydrogel, the gel transition temperature of the thermosensitive chitin is lower than the body temperature, and the injectable mixed hydrogel can be formed into the hydrogel sustained-release drug under the body temperature.

Preferably, a method for preparing the degradable polymer microspheres in step (2) comprises:

(2.1) preparing thermosensitive carboxymethyl chitin into an alkaline carboxymethyl chitin aqueous solution, and dissolving polyethylene glycol with an alkaline solution to obtain a polyethylene glycol solution, and storing at a low temperature;

(2.2) evenly mixing the carboxymethyl chitin aqueous solution with the polyethylene glycol solution at the low temperature under the condition of stirring, heating for physical crosslinking and neutralizing the above reaction system with an acidic solution, then washing and purifying to obtain carboxymethyl chitin microspheres, and performing freeze drying to obtain carboxymethyl chitin porous microspheres; and

(2.3) dissolving local anesthetic and analgesic drug hydrochloride into normal saline or acidic water, adding the carboxymethyl chitin porous microspheres to be soaked, and drying to prepare drug-loaded microspheres, namely, the degradable polymer microspheres containing the local anesthetic and analgesic drugs.

Preferably, the low temperature is 2-30° C., further preferably 4-15° C., the gel transition temperature of the thermosensitive carboxymerthyl chitin is lower than the body temperature, and the injectable mixed hydrogel can be formed into the hydrogel sustained-release drug under the body temperature.

Preferably, the polyethylene glycol has a molecular weight range of 4-30 kDa and a concentration range of 20-50%.

Preferably, the carboxymethyl chitin has an acetylation degree range of 0.72-0.92, a carboxymethyl substitution degree of 0.07-0.23 and a concentration of 0.5-10 wt %.

Preferably, a volume ratio of a carboxymethyl chitin aqueous solution to a polyethylene glycol solution is 1:1-1:20.

The disclosure also provides use of a thermosensitive modified chitin hydrogel local anesthetic-loaded sustained-release analgesia system: preparation and loading of drugs including but not limited to local anesthetic, analgesic, anti-inflammatory and itching relieving effects, thereby prolonging the release time of the drugs so as to achieve the long-acting purpose. Preferably, the system is applied to loading and sustained-release of drugs having an effect of peripheral nerve block or postoperative wound pain control.

Compared with the prior art, the disclosure has the beneficial effects:

(1) according to the disclosure, the thermosensitive modified chitin aqueous solution is directly and evenly mixed with the local anesthetic and analgesic drug aqueous solution, the obtained mixture is injected to a site needing local anesthesia and analgesia so as to quickly form a gel under the body temperature and release the drug, thereby prolonging the analgesia time of the local anesthetic drug in a body, so the preparation process is simple and does not use organic solvents and chemical crosslinking.

(2) In particular, microspheres and local anesthetic-loaded analgesia microspheres are prepared by utilizing thermosensitive carboxymethyl chitin through two water-phase polymer solutions (polyethylene glycol and carboxymethyl chitin dual water phases) so as to finally prepare a novel thermosensitive injectable modified chitin hydrogel and carboxymethyl chitin microsphere composite local anesthetic-loaded analgesia sustained-release system whose drug release speed is lower than that of thermosensitive hydrogel or drug-loaded microsphere alone so as to solve the problem that the existing local anesthetic drug is short in action time, any chemical crosslinking agents and organic solvents are not used in the preparation process, the drug-loaded microspheres and gels have no toxic problems of residual crosslinking agents and organic solvents, the cost is low, and the environment is free from pollution.

(3) The thermosensitive modified chitin material used in the disclosure has good biocompatibility and biodegradability, can be injected and completely fill the irregular wound, and is conducive to long-acting local anesthesia, analgesia, anti-inflammation and itching relieving and promotion of wound healing.

### DESCRIPTION OF THE DRAWINGS

FIG. 1 is a drug cumulative release curve of 2.0% R-HPCH-HA hydrogel sustained-release system represented by 2.0% ropivacaine hydrochloride-loaded in example 2 of the disclosure in a PBS buffer solution, wherein pure drugs (R) with the same drug content and an HPCH carrier (R-HPCH) alone are used as control group.

FIG. 2 shows in-vitro cytotoxicity of an R-HPCH-HA hydrogel sustained-release system loaded with different concentrations of ropivacaine hydrochloride (0.5-5 mM) in example 2 of the disclosure and a pure drug (R) control group on human neuroblastoma cell lines (SH-SY5Y).

FIG. 3A-FIG. 3B show a drug cumulative release curve of three groups of ropivacaine hydrochloride-loaded (R or RPH for short) carboxymethyl chitin micropsheres (CMCH-Ms) having a loading amount of 160 mg/g, drug-loaded hydrogel (HPCH) and a microsphere hydrogel composite drug-loaded sustained-release system (CMCH-Ms/HPCH) in a buffer solution, wherein FIG. 3A is a drug cumulative release curve in a pH5.0 buffer solution, and FIG. 3B is a drug cumulative release curve in a pH7.4 buffer solution.

FIG. 4A-FIG. 4D show an in-vivo anesthetic effect: FIG. 4A shows a maximum pain inhibitory effect ratio (MPE) at different time points after different drug-loaded sustained-release systems are injected around the sciatic nerve of rats; FIG. 4B shows motor block scores at different time points after different drug-loaded sustained-release systems are injected around the sciatic nerve of rats; FIG. 4C shows sensory block time at different time points after different drug-loaded sustained-release systems are injected around the sciatic nerve of rats; FIG. 4D shows motor block time at different time points after different drug-loaded sustained-release systems are injected around the sciatic nerve of rats. The data are represented as mean±standard deviation (SD) (n=8; *P<0.05, **P<0.01).

### DETAILED DESCRIPTION OF PREFERRED EMBODIMENTS

For more easily understanding the disclosure, the specific embodiments of the disclosure will be illustrated in detail below.

Next, the disclosure will be further described in combination with embodiments and drawings for the purpose of helping better understanding the contents of the disclosure, but these embodiments do not limit the protective scope of the disclosure in any manners.

### Example 1: Synthesis of Thermosensitive Injectable Modified Chitin

Thermosensitive chitin derivatives hydroxypropyl chitin and carboxymethyl chitin with low deacetylation degrees, were prepared homogeneously in a sodium hydroxide-urea aqueous solution system by using a homogeneous phase method. A specific preparation method was as follows: 2 g of purified chitin was weighed and dispersed into 100 g of pre-frozen aqueous solution containing 11 wt % sodium hydroxide and 4 wt % urea under the condition of stirring to be frozen for 24 h at −30° C., then the above chitin was taken out and unfrozen at room temperature by mechanically stirring to obtain a dissolved chitin aqueous solution. 11.4 g of 1, 2-propylene oxide was added into the obtained chitin solution (100 g, 2 wt %), this system was stirred at 2° C. so that reactants were evenly mixed, the reactants reacted for 24 h after the temperature was raised to 5° C. and then reacted for 6 h after the temperature was raised to 15° C. Finally, the system was cooled to 2° C., the pH value was adjusted to 7 using 3M hydrochloric acid, the system was dialyzed with deionized water and freeze-dried to obtain white sponge-like hydroxypropyl chitin (HPCH), with a yield of 87%. Based on the ¹H NMR spectrum of HPCH, the degree of acetylation (DA) and the molar degree of substitution of hydroxypropyl (MS) of the product were 0.89 and 0.84, respectively. The viscosity average molecular weight of the product measured by a Ubbelohde viscometer is Mη=410 kDa. The homogeneously synthesized HPCH solution is thermosensitive, and rheological results show reversible sol-gel transition behavior, wherein the gel transition temperature of a 2 wt % HPCH solution is 18° C. By changing the amount of epoxypropane, controlling a molar ratio of epoxypropane to sugar units in the structure of chitin and changing reaction conditions, a series of thermosensitive hydroxypropyl chitins which have different substitution degrees (MS range is 0.53-1.23) and low deacetylation degrees (the acetylation degree range is 0.80-0.95) can be prepared, and their molecular weights range from 5 kDa to 1000 kDa.

Carboxymethyl chitin with a low deacetylation degree was prepared in a sodium hydroxide-urea aqueous solution system by using a homogeneous phase method. 2 g of purified chitin was weighed and dispersed into 100 g of pre-frozen aqueous solution containing 11 wt % sodium hydroxide and 4 wt % urea under the condition of stirring to be frozen for 6 h at −20° C., then the above chitin was taken out and unfrozen at room temperature by mechanically stirring and then frozen and unfrozen twice repeatedly to obtain a dissolved chitin aqueous solution. 5.7 g of sodium chloroacetate was slowly added into the obtained chitin solution (100 g, 2 wt %), mechanical stirring was kept so that reactants can evenly react for 24 h at 5° C., then this system was cooled to 2° C., the pH value was adjusted to 7 using 3M hydrochloric acid, the obtained neutral solution was dropwise added into acetone so that a product was precipitated out. The obtained precipitate was washed with 80% ethyl alcohol to remove salts and urea therein. After washing, the precipitate was dried at 60° C. to obtain white powdered carboxymethyl chitin with a yield of 87%. Based on the ¹H NMR spectrum, the degree of acetylation (DA) and the degree of substitution of carboxymethyl (DS) of the obtained product CMCH were 0.82 and 0.13, respectively. The homogeneously synthesized CMCH solution is thermosensitive and pH-sensitive, can be dissolved under the conditions of a low temperature and alkaline water, and generates gelation transition under the conditions of a raised temperature and reduced alkalinity. By changing the dosage of sodium chloroacetate and controlling the molar ratio of sodium chloroacetate to sugar unit in chitin structure, a series of CMCH products with different substitution degrees (0.07-0.23) were obtained. A series of carboxymethyl chitins with different substitution degrees and low deacetylation degrees (0.72-0.92) can be prepared by changing different molar feed ratios of sodium chloroacetate to saccharide unit and reaction conditions, and their molecular weights range from 5 kDa to 1000 kDa.

Similarly, thermosensitive hydroxyethyl chitin and thermosensitive hydroxybutyl chitin with low deacetylation degrees were prepared in the sodium hydroxide-urea aqueous solution system by using the homogeneous phase method. These chitin derivatives have an acetylation degree of 0.7-0.92 and a molecular weight of 5 kDa-1000 kDa.

### Example 2: Thermosensitive Hydroxypropyl Chitin Hydrogel Local Anesthetic Ropivacaine-Loaded Sustained-Release System

The thermosensitive hydroxypropyl chitin (HPCH with MS: 0.84, DA: 0.89, Tgel: 18° C.) prepared in example 1 was used. 100 mg of hydroxypropyl chitin (HPCH) and 25 mg of hyaluronic acid (HA) were weighed and added into 5 ml of normal saline to be mixed and evenly shaken and then put into a 4° C. refrigerator overnight. After the materials were completely dissolved, a colorless, transparent and flowable viscous liquid (HPCH-HA) was obtained, and then 37.5 mg of local anesthetic drug ropivacaine hydrochloride was added to be dissolved after mixing and shaking to prepare a 0.75% R_{HCL}-HPCH-HA injectable sustained-release hydrogel precursor solution. Before the above precursor solution was injected, a small amount of NaOH solution was added at a low temperature to adjust pH to 5.5-6.0 to obtain a homogeneous white injectable dispersion, and a local anesthetic-loaded ropivacaine hydrogel sustained-release system was formed at 37° C. or in vivo, named as 0.75% R_{HCL}-HPCH-HA (0.75% R-HPCH-HA for short). Similarly, 50 mg, 75 mg, 100 mg or 112.5 mg of local anesthetic drug hydrochloride ropivacaine were used to prepare 1% R_{HCL}-HPCH-HA, 1.5% R_{HCL}-HPCH-HA, 2% R_{HCL}-HPCH-HA, 2.25% R_{HCL}-HPCH-HA injectable local anesthetic-loaded gel sustained-release systems, which were 1.0% R-HPCH-HA, 1.5% R-HPCH-HA, 2.0% R-HPCH-HA and 2.25% R-HPCH-HA for short.

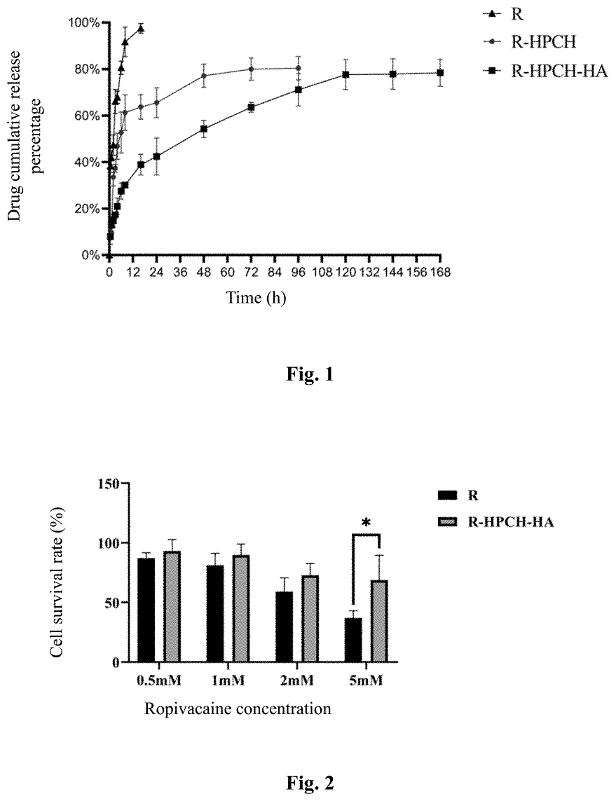

1 ml of the above dispersion (suspension) was sucked with a precision syringe at 4° C. and put into a dialysis bag (molecular weight cut off: 8000-14000 Da), then the dialysis bag was put into a centrifuge tube which was equipped with 30 ml of PBS solution and placed in a 37° C. constant-temperature water-bath shaker that was vibrated at 100 rpm, samples were taken at different time. 2 ml of PBS solution was taken every time to be used as a sample supplemented with 2 mL of fresh PBS solution, the taken sample was analyzed by ultraviolet-visible spectrophotometry, the PBS solution was used as blank control, and a wavelength was detected as 263 nm. According to a standard curve of ropivacaine, the drug concentration of ropivacaine was calculated and a cumulative release curve was plotted. Three samples in each group were set for repetition. FIG. 1 is an in-vitro cumulative release curve of 2.0% R-HPCH-HA hydrogel sustained-release system represented by a 2.0% ropivacaine hydrochloride-loaded, wherein pure drugs (R) with the same drug content and an HPCH carrier (no hyaluronic acid, R-HPCH group) alone with the same drug content are used as control group. It can be seen from FIG. 1 that the pure drug ropivacaine is quickly released in normal saline, and its release rate within 6 hours exceeds 80%, while about 61.2% and 38.9% of ropivacaines in R-HPCH group and R-HPCH-HA group are released within the initial 16 hours, both of which show significant drug sustained-release effects. However, compared with R-HPCH group, R-HPCH-HA group has fewer drug burst release and is more slowly released. Thus, the injectable hydrogel drug delivery system comprising auxiliary component hyaluronic acid is benefit to the long-lasting local analgesia.

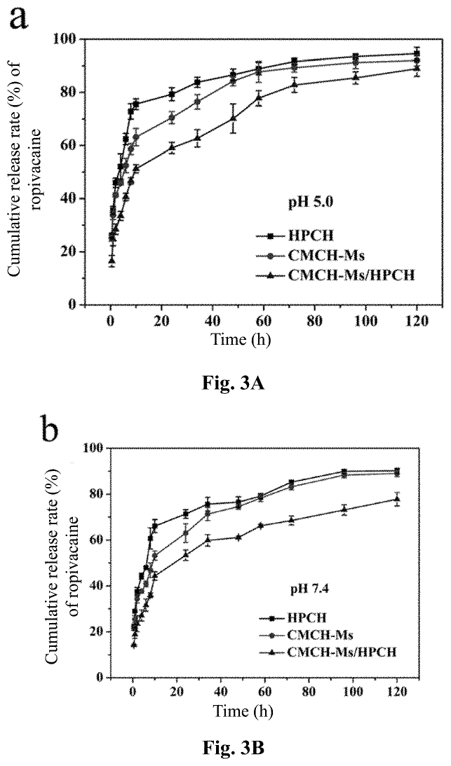

An in-vitro cytotoxicity test was performed on a ropivacaine-containing drug-loaded sustained-release system by using neuroblastoma cell line (SH-SY5Y) cells. Ropivacaine hydrochloride was dissolved into a PBS buffer to prepare a 10 mM ropivacaine solution, and the ropivacaine solution was diluted with a cell culture medium to prepare a cell culture medium with a concentration gradient of 0.5-5 mM in ropivacaine group; cell culture medium solutions containing HPCH2% and HA0.5% as well as a ropivacaine concentration of 10 mM were prepared at a low temperature and gelled at 37° C., and then a cell culture medium containing corresponding gel sustained-release systems and a ropivacaine concentration of 10 mM was prepared. SH-SY5Y cells were inoculated in a 96 well plate at a 10⁴/mL cell density and normally cultured in a constant-temperature incubator for 24 h. Then, the above cell culture mediums containing pure drugs and drug gels were respectively added to be cultured for 24 h. The relative living cell numbers were tested by a Cell Counting Kit 8 (CCK-8) assay (FIG. 2). There was no significant difference in cell viability between two groups under a low concentration (0.5 mM, 1 mM, 2 mM), indicating that HPCH-HA carrier system does not additionally increase cytotoxic response. With the increase of drug concentration, although R-HPCH-HA group had cytotoxicity effect when the concentration was 5 mM, the cytotoxicity response of R-HPCH-HA group was significantly lower than that of pure drug group alone (P<0.05). This means that R-HPCH-HA group showed a significant higher cell viability than R_{HCL} group at the higher concentrations (5 mM: 59.1% vs 37.2%. P<0.001, n=8/group). This suggested that using HPCH-HA as a controlled release system may protect local tissues against ropivacaine-induced cytotoxicity.

### Example 3: Preparation of Carboxymethyl Chitin Porous Microspheres and Drug-Loaded Microspheres Via a Solvent-Free Green Method

10 ml of 2 wt % sample solution was prepared by using the powdered thermosensitive carboxymethyl chitin CMCH (DS 0.13 and DA 0.82) synthesized in example 1 and a 1 mol/L sodium hydroxide solution and stored at 2° C. 50 mL of 30 wt % polyethylene glycol (PEG) solution was prepared by using PEG-10k (polyethylene glycol with a molecular weight of 10000) and a 1 mol/L sodium hydroxide solution. In an ice water bath under mechanical stirring, 10 mL of the CMCH solution was slowly added into the 50 mL of 30 wt % PEG solution and stirred for half an hour. Then, a 60° C. oil bath was used to replace the ice water bath, and meanwhile the system was neutralized to be neutral with 1.0 M HCl. Neutralization and heating were performed for physical crosslinking so that microspheres were solidificated to form the sable microspheres for 20 min without using any chemical crosslinking agents. Then these carboxymethyl chitin microspheres were repeatedly washed with deionized water and freeze-dried to obtain carboxymethyl chitin porous microspheres. Change in a volume ratio of the CMCH aqueous solution to the polyethylene glycol solution of (1:10-7:10), change in the molecular weight (from 6k to 30k) of PEG, change in the concentration (20-50%) of PEG, change in degrees of substitution (DS between 0.07-0.23) of CMCH samples with low degree of deacetylation (DA between 0.72-0.92) and the concentration (0.5-10 wt %) of CMCH and a volume ratio (1:1-1:20) of the CMCH solution to the PEG solution do not affect the above preparation.

Ropivacaine hydrochloride (R) powder was dissolved into a 0.1 M HCl solution to prepare different concentrations of drug solutions (1-80 mg/mL). 0.1 g of lyophilized carboxymethyl chitin porous microspheres were soaked into 0.5-5 mL of ropivacaine solutions with different concentrations for a certain time such as 2, 4, 6, 8, 12, 24 h, and then the ropivacaine drug was loaded. The above mixture was centrifuged for 5 min at a rotation speed of 3000 rpm, and the concentration of ropivacaine in the supernatant was determined by UV-vis spectrophotometer at 262 nm and quantitatively analyzed by standard curves to calculate the amounts mₛᵤₚ of drugs in which the microspheres were not loaded if there was a flowing liquid; if there is no flowing liquid, mₛᵤₚ was 0. Then the drug-loaded microspheres were obtained by drying it in an oven

The calculation formula of the drug loading capacity (LC) is

$$L C = \frac{{m}_{add} - {m}_{sup}}{{m}_{c}}$$

wherein, m_{add} is the weight of the initially added ropivacaine and m_{c} is the weight of the dried drug-loaded microspheres.

The drug loading amount of ropivacaine hydrochloride in the carboxymethyl chitin porous microspheres was increased with the increase of soaking time. When the soaking time reached 24 h, the drug loading amount was basically saturated, and the drug loading amount was increased with the increase of ropivacaine concentration. For example, when the R concentration of ropivacaine hydrochloride was 8 mg/mL and the soaking time was 24 h, the ropivacaine hydrochloride-loaded carboxymethyl chitin porous microspheres (R-loaded CMCH-Ms) having a microsphere loading amount of 160 mg/g were prepared. Preparation of other drug-loaded microspheres such as ropivacaine-loaded PLGA microspheres needs to use an organic solvent.

### Example 4: Preparation of an Injectable Microsphere Hydrogel Composite Drug-Loaded Sustained-Release System

10 mg of dried R-loaded CMCH-Ms (drug LC 160 mg/g) prepared in Example 3 was added into 1 mL of 3% HPCH (MS 0.89, DA 0.94) solution at 4° C. and evenly mixed to obtain an injectable microsphere hydrogel composite drug-loaded precursor solution which rapidly formed a gel at 37° C. or in vivo. Meanwhile, a hydroxypropyl chitin gel (R-loaded HPCH) loaded with ropivacaine hydrochloride was used as control group by adding the same amount of ropivacaine as loaded in CMCH-Ms/HPCH into 1 mL of 3% HPCH solution at 4° C. and evenly mixing so as to form the gel at 37° C.

In an in-vitro drug release experiment, 10 mg of R-loaded CMCH-Ms, 1 mL of R-loaded HPCH gel and 1 mL of R-loaded CMCH-Ms/HPCH gel were respectively weighed and put into dialysis bags (molecular weight cut off: 8-14 kDa), then the dialysis bags were put into centrifuge tubes equipped with 10 mL of pH 5.5 and pH 7.4 buffer solutions. The centrifuge tubes were placed in a water bath shaker at 37° C. to be vibrated at the speed of 70 rpm, 2 ml of release medium was taken from the centrifuge tube at a predetermined time point and 2 ml of fresh buffer solution was supplemented. The absorbance of ropivacaine hydrochloride R concentration in the taken release medium was measured by a UV-VIS Spectrometer at 262 nm.

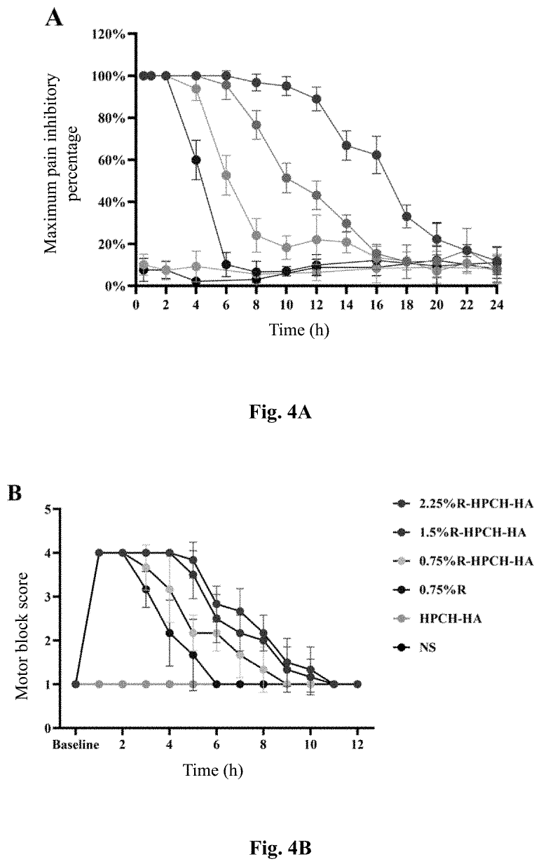

According to the standard curve of ropivacaine, the drug concentration of ropivacaine was calculated and the cumulative percentage of drug release was calculated. Each group of experiments was repeated three times in parallel, and test results were mean±standard deviation (SD) values. It can be seen from FIG. 3A-FIG. 3B that at pH 5.0, the initial drug release of R-loaded CMCH MS/HPCH group at 1 h is 24.7±2.4%, and the initial drug release of R-loaded CMCH MS group and R-loaded HPCH group at 1 h are 33.7±1.6% and 35.5±1.5%, respectively. At pH 7.4, the initial drug release of R-loaded HPCH group and R-loaded CMCH-Ms group at 1 h are 28.9±0.14% and 25.4±1.5%, respectively, while the initial drug release of the microsphere hydrogel composite drug delivery system R-loaded CMCH-Ms/HPCH group is 18.8±1.7%. Therefore, the microsphere hydrogel composite drug delivery system shows an obvious drug sustained release effect, has fewer drug burst release and slower release compared with the thermosensitive hydrogel or microspheres as a carrier alone, and exhibits long-acting sustained release. Similar sustained-release effect can be obtained by using other drug-loaded microspheres such as ropivacaine-loaded PLGA mivrospheres instead of carboxymethyl chitin drug-loaded microspheres.

### Example 5: Evaluation on Long-Acting Sustained-Release Anesthesic and Analgesic Effect and Safety in Animals

In order to evaluate the in-vivo analgesic effect of the drug-loaded sustained-release system, an ultrasound-guided sciatic nerve block model was used. Various sustained-release injection formulations containing ropivacaine were injected around the sciatic nerve of the right hind limb of SD rats under the guidance of ultrasound, and the nerve blocking effect was observed. The rats were anesthetized with 2% isoflurane and placed in a prone position. Under the guidance of ultrasound, a 23G needle was vertically inserted into the medial femur to reach between the two layers of fascia of the middle gluteal muscle, and 0.5 mL of drug injection formulations were injected around the proximal trunk of the sciatic nerve. Then, the sensory and motor block of the right hind limbs of the rats were evaluated by a hot plate method and motor block 4-point method, respectively. The experiment was divided into 6 groups: (1) 2.25% R-HPCH-HA, (2) 1.5% R-HPCH-HA, (3) 0.75% R-HPCH-HA, (4) 0.75% R, (5) HPCH-HA, (6) NS (normal saline), with 6 rats in each group.

Operation with hot plate method: the rats were placed on a preheated metal surface (55±0.5° C.), which was surrounded by a cylindrical Plexiglas wall (20 cm high and 28 cm in diameter) to limit the movement of the rats. The rats were put and timed immediately. When researchers observed that there was one of the following pain responses: hind paw licking, hind paw shaking/flutter, jumping in the right hind limb of the rats, indicating that the feeling of pain appeared, the timing was stopped immediately and the rats were taken out from the hot plate. This time was recorded as the basic thermal latency (baseline) time of rats (tested 2 days before the beginning of the experiment), which was generally about 3-5 s, and the rats with too short or too long baseline time were excluded. In order to prevent tissue injury, if the rats still have no pain response after 20 s, the rats were taken out and the time was recorded as 20 s. Each measurement was performed at an interval of 5 minutes so as to prevent irritation sensitization. The evaluation began 15 min after the injection of the drug, and then the test was performed every 2 hours. If the 3 measurements of rats all reached a baseline level, measurement was performed every 4 hours until all the rats were completely tested. All observation tasks were completed by three independent observers. The test data were expressed as percentage of maximum possible effect (MPE %) with the calculation formula as follows:

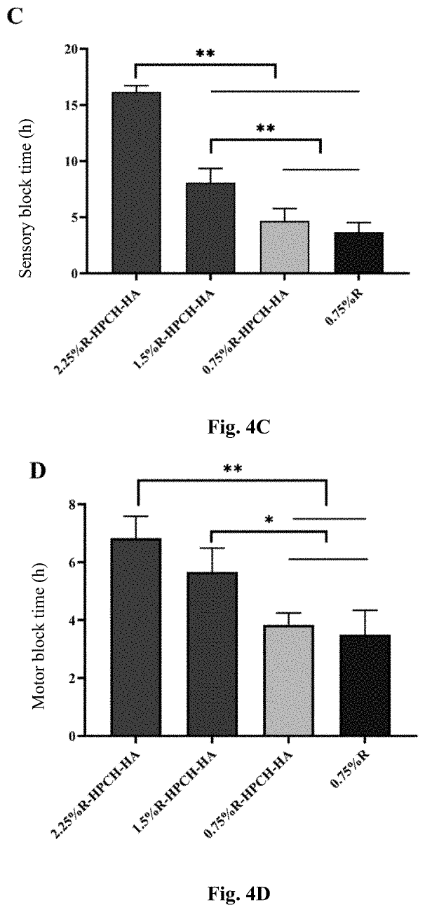

MPE %=(Lt−Bt)/(Ct−Bt)×100%

wherein, Bt represents baseline time of the rat; Lt represents heat latency time of the rats at different time points; Ct represents a maximum allowable heat latency time (cut-off time) of the rat, namely, 20 s. As shown in FIG. 4A, all the rats treated with the groups containing drug ropivacaine reached the maximum cut-off value (20 s) at 15 min postinjection, showing that the application of HPCH-HA sustained-release system does not affect the onset time of the drug. The MPE value of the 0.75% R pure drug control group came back to the normal baseline after 6 hours, while the average MPE % value of R-HPCH-HA groups is prolonged in a dose-dependent manner. The effective analgesic effect of a sustained-release drug formulation is defined as a time span (sensory block time) required for recovering an MPE value after injection from 100% to 50%. The 50% MPE analgesia durations (sensory block time in FIG. 4C) for 2.25% R-HPCH-HA group and 1.5% R-HPCH-HA group are 16.2 h and 8.1 h, respectively, which are significantly longer than that of R pure drug control group (P<0.01). The carrier alone itself has no nerve block effect. Motor block and sensory block of the rats were also detected, the effect of dyskinesia was scored, that is, the motor block 4-point method (1 score indicates that the rats' movement is completely normal; 2 score indicates that the rats' foot dorsiflexion dysfunction cannot fully open their toes when lifting the rat tail; 3 sore indicates that the rats' plantar flexion disorder cannot fully open their toes when lifting the rat tail; 4 score indicates that the rats have completely lost the function of dorsal flexion and plantar flexion and have gait disorder). The blockade time (i.e., the ending of an effective motor blockade) was defined as the earliest time point when the score was <2. The duration of motor blockade was defined as the time period from drug administration to the blockade time. As shown in FIG. 4D, the motor block time of 2.25% R-HPCH-HA group and 1.5% R-HPCH-HA group were 6.8 h and 5.6 h respectively. Compared with 0.75% R pure drug control group, there is a statistical significance for prolonged motor block time (P<0.01). After 12 hours, the motor function scores of all the rats returned to be normal. Rats treated with only pure polymer HPCH-HA carrier or normal saline did not show significant changes of sensory and motor functions at any time point after injection.

von Frey test: the von Frey test were used to evaluate the mechanical sensitivity. The paw withdrawal threshold (PWT) was measured to assess mechanical sensitivity using different forces of the von Frey filaments (0.6, 1, 1.4, 2, 4, 6, 8, 15, 26, 60 g). PWTs were determined by using a version of the up-down method, and were measured before and at 6 hours, 12 hours, and 24 hours after injection. The mechanical PWTs measured at 6, 12, and 24 hours after injection of 2.25% R-HPCH-HA were significantly higher than those in other groups. PWTs at 6 and 12 hours after 1.5% R-HPCH-HA treatment were significantly higher than those after 0.75% R-HPCH-HA or 0.75% R_{HCL} treatment. At 6 hours postinjection, PWT in 0.75% R-HPCH-HA group was significantly higher than that in 0.75% R_{HCL} group.

Tissue inflammation and neurotoxicity assessment: HPCH-HA residual gel injected into rats was adhered to tissues near the sciatic nerve, and almost no gel was observed on day 14 and basically completely degraded. The HE tissue staining showed that a 2.25% R-HPCH-HA implantation site had moderate inflammation on day 7, while other ropivacaine containing groups and carrier alone group only had mild inflammation, and after 14 days, the inflammatory responses of the ropivacaine-containing groups and the carrier alone group were all alleviated, indicating that the inflammatory response caused by HPCH-HA as the ropivacaine carrier has a certain reversibility. We did not observe an infiltration of inflammatory cells into the epineurium in any group, indicating that HPCH-HA as the ropivacaine carrier did not cause inflammatory response to the sciatic nerve. On day 7 and day 14 after injection, sciatic nerve tissues were tested by transmission electron microscope (TEM), toluidine blue (TB) staining and terminal deoxynucleotidyl transferase-mediated dUTP nick-end labeling (TUNEL) assays. The test results show that the axon and myelin sheath structures exhibit orderly arranged fibers and uniform myelin sheath structures in each group. There was no significant difference in axon density and abnormal myelin sheath percentage between drug-treated and saline groups, and there was no significant difference in TUNEL positive cell test results, indicating that there was no significant apoptosis change in each group. The above results show that the ropivacaine-containing sustained-release system is degradable in the body, slightly affects the structure of nerve fibers and has no obvious structural damage. After injection of ropivacaine-containing formulations for 7 days and 14 days, there was no significant difference in aspartate aminotransferase (AST), blood urea nitrogen (BUN) and creatinine (CR) among rats in each group, which were within a normal range. Therefore, it shows that the ropivacaine-containing HPCH-HA sustained-release system has no significant effect on liver and kidney functions of rats.

The concentration of the thermosensitive hydroxypropyl chitin replaces the above 2% from 0.5-5%, the concentration of the local anesthetic drug is within a range of 0.1-5%, and the addition concentration of hyaluronic acid is between 0.1% and 1%. The local anesthetic and analgesic drug are a mixture selected from any one or more of procaine, bupivacaine, levobupivacaine, tetracaine, ropivacaine, etidocaine, articaine, lidocaine, mepivacaine, prilocaine or oxethazaine, and their salts and prodrugs, which can replace the ropivacaine in the above experiment to obtain similar long-acting sustained-release results. Since 1-3% thermosensitive hydroxypropyl chitin and 0.2-0.8% hyaluronic acid auxiliary components are contained, the local anesthetic and analgesic drug selected from 0.5-3% ropivacaine hydrochloride has a better long-acting sustained-release effect.

At last, it should be noted that the above embodiments are only for illustrating the technical solution of the disclosure but not limiting the protective scope of the disclosure. Although the disclosure is described in detail with reference to preferred embodiments, those skilled in the art should understand that modifications or equivalent replacements can be made to the technical solution of the disclosure without departing from the essence and scope of the technical solution of the disclosure.

We claim:

1. A thermosensitive modified chitin hydrogel local anesthetic-loaded sustained-release analgesia system, wherein the local anesthetic-loaded sustained-release analgesia system is an injectable mixed hydrogel prepared by mixing thermosensitive modified chitin with a local anesthetic and analgesic drug, the gel transition temperature of the thermosensitive modified chitin is lower than a body temperature, the concentration of the thermosensitive modified chitin in the injectable mixed hydrogel is 0.5-5% by mass, and the concentration of the local anesthetic and analgesic drug in the injectable mixed hydrogel is 0.1-5% by mass.

2. The thermosensitive modified chitin hydrogel local anesthetic-loaded sustained-release analgesia system according to claim 1, wherein the thermosensitive modified chitin is a combination of any one or more of thermosensitive hydroxypropyl chitin, thermosensitive hydroxyethyl chitin or thermosensitive hydroxybutyl chitin.

3. The thermosensitive modified chitin hydrogel local anesthetic-loaded sustained-release analgesia system according to claim 1, wherein the local anesthetic-loaded sustained-release analgesia system also contains hyaluronic acid dissolved into the injectable mixed hydrogel to serve as an auxiliary ingredient.

4. The thermosensitive modified chitin hydrogel local anesthetic-loaded sustained-release analgesia system according to claim 1, wherein the at least one drug substance is present in an amount sufficient to provide long lasting local analgesia, local anesthesia.

5. The thermosensitive modified chitin hydrogel local anesthetic-loaded sustained-release analgesia system according to claim 4, wherein the local anesthetic and analgesic drug is an amino amide anaesthetic, an amino ester anesthetics and mixtures thereof, selected from any one or more of procaine, bupivacaine, levobupivacaine, tetracaine, ropivacaine, etidocaine, articaine, lidocaine, mepivacaine, prilocaine and oxethazaine, and their salts and prodrugs.

6. The thermosensitive modified chitin hydrogel local anesthetic-loaded sustained-release analgesia system according to claim 5, wherein the concentration of local anesthesia ranging 0.5-3 wt %, the local anesthesia selected from bupivacaine or ropivacaine.

7. The thermosensitive modified chitin hydrogel local anesthetic-loaded sustained-release analgesia system according to claim 1, wherein the local anesthetic-loaded sustained-release analgesia system also contains degradable polymer microspheres loaded with local anesthetic and analgesic drugs, which are evenly dispersed in the injectable mixed hydrogel.

8. The thermosensitive modified chitin hydrogel local anesthetic-loaded sustained-release analgesia system according to claim 7, wherein the degradable polymer microspheres are thermosensitive carboxymethyl chitin porous microspheres.

9. A preparation method of a thermosensitive modified chitin hydrogel local anesthetic-loaded sustained-release analgesia system, comprising the following steps:
(1) dissolving thermosensitive hydroxypropyl chitin and hyaluronic acid into normal saline or alkaline water below the gel transition temperature to obtain solution A;
(2) dissolving local anesthetic and analgesic drug hydrochloride into normal saline or acidic water to obtain solution B; and
(3) mixing the solution A with the solution B at a low temperature to prepare a mixed hydrogel precursor solution, and adjusting pH to 5-6 to obtain a homogenous injectable mixed hydrogel, namely, the local anesthetic-loaded sustained-release analgesia system;
wherein the at least one drug substance is an amino amide anaesthetic, an amino ester anesthetics and mixtures thereof, selected from the group comprising bupivacaine, levobupiyacaine, lidocaine, mepivacaine, prilocaine, ropivacaine, articaine, trimecaine, procaine, tetracaine and their salts and prodrugs; and
the local anaesthetic agent is present in an amount of from 0.1 wt % to about 5 wt %.

10. The method of claim 9, wherein the thermosensitive modified chitin is thermosensitive hydroxypropyl chitin, hydroxyethyl chitin, hydroxybutyl chitin or their combination.

11. The method of claim 10, wherein the thermosensitive modified chitin is thermosensitive hydroxypropyl chitin and thermosensitive hydroxypropyl chitin is present in an amount of from 0.5 wt % to about 5 wt %.

12. The method of claim 11, adjusting the pH value of the therapeutic hydrogel precursor solution to 5.5-6.0.

13. The method of claim 11, thermosensitive hydroxypropyl chitin is present in an amount of from 1 wt % to about 3.6 wt %.

14. A preparation method of a thermosensitive modified chitin hydrogel/microsphere composite local anesthetic-loaded sustained-release analgesia system, comprising the following steps:
(1) dissolving thermosensitive modified chitin into normal saline or alkaline water below the gel transition temperature to obtain solution A;
(2) preparing degradable polymer microspheres containing local anesthetic and analgesic drugs; and
(3) evenly mixing the degradable polymer microspheres containing the local anesthetic and analgesic drugs in the solution A at a low temperature to prepare an injectable mixed hydrogel, namely, the local anesthetic-loaded sustained-release analgesia system, wherein the low temperature is lower than the gel transition temperature of the thermosensitive modified chitin;
wherein the at least one drug substance is an amino amide anaesthetic, an amino ester anesthetics and mixtures thereof, selected from the group comprising bupivacaine, levobupiyacaine, lidocaine, mepivacaine, prilocaine, ropivacaine, articaine, trimecaine, procaine, tetracaine and their salts and prodrugs; and
the local anaesthetic agent is present in an amount of from 0.1 wt % to about 5 wt %.

15. The method of claim 14, wherein the thermosensitive modified chitin is thermosensitive hydroxypropyl chitin, hydroxyethyl chitin, hydroxybutyl chitin or their combination.

16. The method of claim 14, wherein the thermosensitive modified chitin is thermosensitive hydroxypropyl chitin and thermosensitive hydroxypropyl chitin is present in an amount of from 0.5 wt % to about 5 wt %.

17. The preparation method of the thermosensitive modified chitin hydrogel/microsphere composite local anesthetic-loaded sustained-release analgesia system according to claim 14, wherein the step (2) specifically comprises:
(1) preparing thermosensitive carboxymethyl chitin into an alkaline carboxymethyl chitin aqueous solution, and dissolving polyethylene glycol with an alkaline solution to obtain a polyethylene glycol solution, and storing at a low temperature;
(2) evenly mixing the carboxymethyl chitin aqueous solution with the polyethylene glycol solution under the condition of stirring, heating for physical crosslinking and neutralizing the above reaction system with an acidic solution, then washing and purifying to obtain carboxymethyl chitin microspheres, and performing freeze drying to obtain carboxymethyl chitin porous microspheres; and
(3) dissolving local anesthetic and analgesic drug hydrochloride into normal saline or acidic water, adding the carboxymethyl chitin porous microspheres to be soaked, and drying to prepare drug-loaded microspheres, namely, the degradable polymer microspheres containing the local anesthetic and analgesic drugs.

18. Use of the thermosensitive modified chitin hydrogel local anesthetic-loaded sustained-release analgesia system according to claim 1 for a drug for long-acting local anesthesia, analgesia, anti-inflammation and itching relieving.

19. Use of the thermosensitive modified chitin hydrogel local anesthetic-loaded sustained-release analgesia system according to claim 1 for a drug for block of peripheral nerves or control of postoperative wound pain.
